(19) United States
(12) Patent Application Publication
(10) Pub. No.: US 2013/0172757 A1
(43) Pub. Date: Jul. 4, 2013

(54) MULTIPLE TRANSDUCER AUTOMATIC INITIATION

(71) Applicants: Sigmund Frigstad, Trondheim (NO); Haakon Hoye, Horten (NO)

(72) Inventors: Sigmund Frigstad, Trondheim (NO); Haakon Hoye, Horten (NO)

(73) Assignee: GENERAL ELECTRIC COMPANY, Schenectady, NY (US)

(21) Appl. No.: 13/343,130

(22) Filed: Jan. 4, 2012

(51) Int. Cl. A61B 8/14

(52) U.S. Cl. 600/459

(57) ABSTRACT

A first transducer and a second transducer provided by an ultrasound probe are spaced so as to be alternately coupled to tissue overlying an object to be imaged. The emission of ultrasound pulses by one of the first transducer and the second transducer is automatically initiated based on positioning of said one of the first transducer and the second transducer to the tissue overlying the object to be imaged.

### BACKGROUND

Ultrasound or ultrasonography is a medical imaging technique that utilizes high-frequency (ultrasound) waves and their reflections. Such ultrasound waves are directed into a patient's anatomy using a handheld probe having an ultrasound transducer. Some handheld probes include multiple transducers that emit and sense different frequencies of ultrasound waves. Determining which of the multiple transducers to activate and manually activating the correct transducer may sometimes be confusing, may consume valuable time and may undesirably detract from a caretaker's focus on care of the patient.

### BRIEF DESCRIPTION OF THE DRAWINGS

FIG. 1 is a schematic illustration of an example multiple transducer automatic initiation system.

FIG. 2 is a flow diagram of an example method that may be carried out by the system of FIG. 1.

FIG. 3 is a schematic illustration of another example multiple transducer automatic initiation system.

FIG. 4 is a flow diagram of an example method that may be carried out by the system of FIG. 3.

### DETAILED DESCRIPTION OF THE EXAMPLE EMBODIMENTS

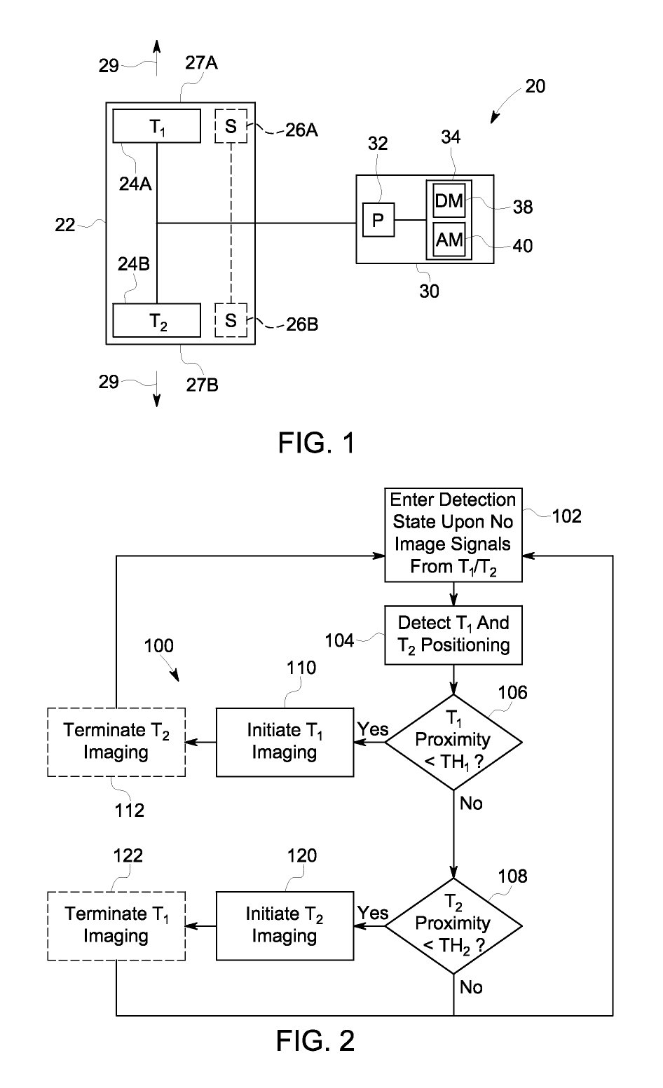

FIG. 1 schematically illustrates an example multiple transducer automatic initiation system 20. As will be described hereafter, system 20 detects which of two transducers are positioned and closely coupled against or in close proximity to tissue overlying an object to be imaged and automatically initiates imaging for the detected transducer. As a result, the correct transducer will automatically be activated, allowing the caretaker to focus on care of the patient.

As shown by FIG. 1, system 20 comprises handheld probe 22, transducers 24A, 24B (collectively referred to as transducers 24), sensors 26A, 26B (collectively referred to as sensors 26), and controller 30. Handheld probe 22 comprises a handheld instrument by which a person may manually alternately position one of transducers 24 against tissue overlying the object that is to be imaged. Such imaging may be directed at an object such as an anatomical structure or at an object such as blood flow. Handheld probe 22 supports transducers 24 along different external surfaces 27A, 27B or in different directions such that transducer 24 may only be alternately coupled to skin or other tissue overlying the object to be imaged. In the example illustrated in FIG. 1, probe 22 supports transducers 24 at opposite ends of probe 22, wherein transducers 24 face in opposite directions (as indicated by arrows 29) and emit ultrasound pulses along coincident or parallel centerlines in such opposite directions.

Transducers 24 comprises quartz crystals, piezoelectric crystals, that change shape in response to the application electrical current so as to produce vibrations or sound waves. Likewise, the impact of sound or pressure waves upon such crystals produce electrical currents. As a result, such crystals are used to send and receive sound waves. Each transducer-24 may additionally include a sound absorbing substance to eliminate back reflections from the probe itself and an acoustic lens to focus emitted sound waves.

Transducers 24A and 24B are configured to emit and receive sound waves having different ranges or scopes of characteristics. For example, in one implementation, transducers 24A and 24B may emit and receive different ranges of sound wave frequencies. In other implementations, transducer 24A and 24B may have other different characteristics or different capabilities. Because transducers 24A and 24B are supported and carried by a single handheld probe 22, a caretaker has immediate access to both types of transducers 24A and 24B and may choose one of the types by selectively positioning the appropriate transducer against the tissue overlying the object to be imaged.

In one implementation, transducer 24A may comprise a linear array transducer while transducer 24B comprises a sector array transducer. In such an example implementation, transducer 24A has a frequency range of 6-10 MHz, with the array having dimensions of approximately 3 to 4 cm×0.5 cm. In contrast, transducer 24B has a frequency range of 3-5 MHz, with the array having dimensions of 2 cm×1.5 cm. Transducer 24A offers greater resolution for viewing or imaging more superficial anatomy such as veins. Transducer 24B offers greater depth or penetration for viewing or imaging such internal anatomy as a heart, liver or the like.

In other implementations, one of transducer 24 may alternatively comprise a different type of transducer. For example, one of transducers 24 may alternatively comprise a curved array transducer which has a frequency and dimensions that facilitate a larger sonographic window. In some implementations both of transducers 24 may be replaced with other types of transducers having different features or characteristics.

Sensors 26 comprise devices configured to detect positioning of their associated transducers 24 with respect to the tissue that overlies the object to be imaged. In the example illustrated, sensor 26A detects the positioning of transducer 24A while sensor 26B detects the positioning of transducer 24B. In one implementation, sensors 26 detect physical coupling of the associated transducer 24 to tissue overlying the object to be imaged. For purposes of this disclosure, the term “physical coupling” refers to the transducer or a surface of the probe overlying the transducer being brought into engagement or contact with the tissue (such as skin) overlying the object to be imaged. In one implementation, sensor 26 may comprise a pressure sensor, such as a capacitive layer that is placed in front of an acoustic lens of the transducer, behind the crystals of the transducer or next to the transducer or acoustic lens. For example, such a capacitive sensor may be formed as a ring or a multiple point sensor on a structure on the front of a nose or end of the handheld probe 22. In another implementation, sensor 26 may comprise optical sensors, such as an infrared sensor LED diode sensors that compare reflected optical signal pattern to a reference signal obtained when scanning an air. In yet other implementations, sensors 26 may comprise thermistors configured to detect a rapid change in temperature, such as when the sensor is brought into contact with the tissue.

In still other implementations, sensors 26 comprise proximity sensors which detect a distance that the associated transducer 24 is spaced from the tissue overlying the target or object to be imaged. As a result, sensor 26 may be used to trigger activation of the associated transducer (as well as other processes) prior to the transducer becoming physically coupled to the tissue, allowing a greater amount of time for the activation of the associated transducer or allowing the transducer itself to be activated earlier in time for more prompt imaging.

As indicated by the broken lines illustrating sensors 26, in one implementation, such sensors may 26 may alternatively be provided by transducers 24 themselves. In particular, transducers 24 may be controlled so as to emit short bursts of ultrasound at regular intervals, wherein the echoes from such pulses are analyzed by the controller 30 to determine whether the particular transducer emitting such pulses is physically coupled to the tissue or to determine a spacing or proximity of the transducer emitting the pulses from the tissue. Echoes from pulses penetrating tissue or anatomy have different characteristics as compared echoes from pulses reflected from air. The distance that the ultrasound waves must travel prior to impinging tissue also impacts the characteristics of such echoes, allowing controller 30 to determine a distance between the transducer 24 emitting such pulses and the tissue overlying the target or object to be imaged.

Controller 30 is in communication with transducers 24 and sensors 26 (where provided separately from transducers 24). In one implementation, controller 30 is provided as part of a host or monitor remote from probe 22 that communicates with probe 22 in a wired or wireless fashion. In another implementation, controller 30 is provided as part of or housed within probe 22. Controller 30 generates control signals directing the operation of transducers 24 and sensors 26. Controller 30 activates sensors 24 based upon information from sensors 26 (or from transducer positioning information from transducers 24). Controller 30 comprises processing unit 32 and memory 34 which includes detection module 38 and activation module 40.

Processing unit 32 comprises one or more processing units configured to generate control signals in response to sensed signals received by sensors 26 (or transducers 24 serving at sensors 26) according to instructions contained in memory 34. One implementation, memory 34 comprises a non-transient computer-readable medium providing persistent storage for such instructions or code. In one implementation, memory 34 may comprise software. In another implementation, memory 34 and parts of processor or processing unit 32 may comprise an application-specific integrated circuit (ASIC).

Modules 38 and 40 each comprise a non-transient computer readable program or code stored in memory 34 and configured to direct processor 32 to carry out the process or method 100 shown in FIG. 2. Detection module 38 directs processor 32 in the detection of the positioning of transducers 24 to determine when one of transducers 24 is sufficiently close to tissue overlying the target or object to be imaged such that the transducer may be activated for full scanning or imaging. Activation module 40 directs processor 30 to generate control signals to activate one of the transducers 24.

As indicated by step 102 in FIG. 2, during operation of system 20, in response to imaging signals no longer being received for a predetermined period of time (indicating that neither of transducers 24 is physically coupled to tissue overlying target or object being imaged), system 20 will enter into a transducer position detection state. In the position detection state, detection module 38 directs processor 32 to detect the position of transducers 24 with respect to the tissue overlying the target or object to be imaged. In one implementation, the predetermined period of time or delay before entering into the detection state is such that a temporary decoupling of a transducer during imaging to simply reposition the same transducer against the tissue will not trigger the detection state.

As indicated by steps 104, 106 and 108, once system 20 has entered the detection state, detection module 38 directs processor 32 and sensors 26 (transducers 24 when serving as transducers 26) to continuously or periodically at predefined intervals detect the positioning of transducers 24 until one of transducers T1 or T2 is detected as being within a predefined proximity of tissue overlying target or object to be imaged. In the example illustrated, processor 32 determines whether transducers 24A and 24B have a proximity to the skin or tissue that is less than predefined thresholds TH1 and TH2, respectively, which may be stored in memory 34. In one implementation, both of such thresholds TH1 and TH2 may be established at a value such that the associated transducers must be physically coupled to the tissue to satisfy the thresholds. In another implementation, both of such thresholds TH1 and TH2 may be established at a value such that the thresholds are satisfied when the transducers 24 are spaced from, but within a predefined distance of the tissue. In some implementations, the thresholds TH1 and TH2 for the different sensors 24A and 24B, respectively, may be different from one another. For example, system 20 may activate one of sensors 24 prior to being placed in contact or physical coupling with the tissue while activating the other of sensors 24 only after the other sensor 24 is in contact or physical coupling with the tissue. By way of another example, system 20 may activate sensor 24A when at a first distance from the tissue while activating sensor 24B when at a second greater distance from the tissue.

Activation module 40 directs processor 32 in the activation of transducers 24 upon processor 30 to determining that one of transducers 24 satisfies the associated distance threshold. As indicated by step 110, in response to processor 32 determining that transducer 24A is within the predefined proximity to the tissue overlying the target or object to be imaged (having a proximity<TH1), activation module 40 automatically initiates imaging by transducer 24A. In particular, rather than being in a dormant state or power saving mode and rather than periodically emitting short bursts of ultrasound pulses for proximity detection, transducer 24A emits ultrasound pulses at frequencies and for periods of time appropriate to image the target or object underlying the tissue at the appropriate depth.

As indicated by step 112, in some implementations, where transducer 24B may still be emitting ultrasound pulses from a previous imaging operation, activation module 40 may further direct processor 32 to generate control signals to terminate the emission of such imaging ultrasound pulses from transducer 24B. In other implementations, step 112 may be omitted.

As indicated by steps 120 and 122, activation module 40 directs processor 32 in a similar fashion with respect to transducer 24B in response to processor 32 determining that transducer 24B is within the predefined proximity to the tissue overlying the target or object to be imaged (having a proximity<TH2). In each of steps 110, 112, 120, 122, such initiation of imaging is automatic, requiring no manual actuation of any controls on a host or on probe 22. As a result, a caretaker merely needs the position a selected one of transducers 24 towards or against the tissue overlying the target or object to be issued to activate the selected transducer. Should the caretaker decide to switch to the other transducer, the caretaker merely needs to reposition probe 22 to alternatively reposition the other transducer towards or against the tissue overlying the target or object to be imaged. Once again, the newly selected transducer will be automatically activated for use.

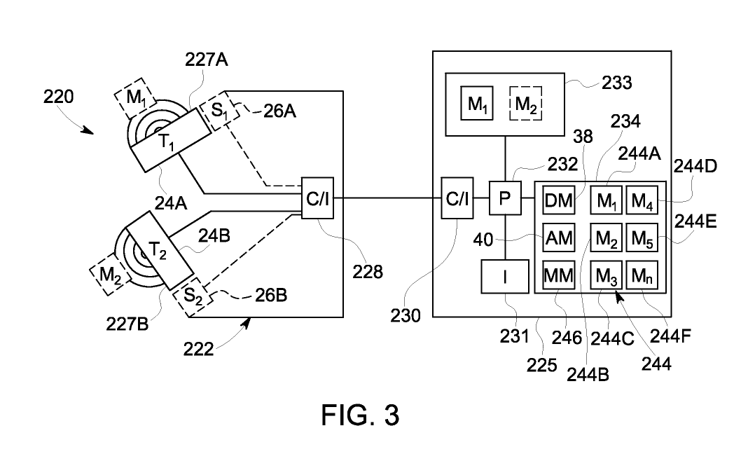

FIG. 3 schematically illustrates multiple transducer automatic initiation system 220, an example implementation of system 20 described above. Like system 20, system 220 detects which of two transducers are positioned or closely coupled against or in close proximity to tissue overlying target or object to be imaged and automatically initiates target or object imaging for the detected transducer. As a result, the correct transducer will automatically be activated, allowing the caretaker to focus on care of the patient. As will be described hereafter, system 220 additionally automatically initiates or prompts authorization for different available modes of operation (also known as presets) based upon which of the transducers is detected as being in sufficient proximity to the tissue overlying the target or object to be imaged.

System 220 comprises probe 222 and host 225. Probe 222 is similar to probe 22 (shown in FIG. 1) except that probe 222 supports transducers 24 along or adjacent to surfaces 227A, 227B which are oblique to one another. As with probe 22, probe 222 supports such transducers 24 at positions and orientations such that transducers 24 may not be simultaneously placed in contact or physical coupling with tissue overlying an target or object to be imaged. In other words, transducers 24 are positioned to inhibit simultaneous sensing of the same target or object with both transducers. In the example illustrated, because transducers 24 are positioned at oblique angles with respect to one another, rather than at opposite ends, communication interface 228 may be positioned at an unused end without interfering with those surfaces of probe 222 used for manual gripping and manipulation of probe 222. In other examples, transducers 24 may be supported other angles or other positions. For example, in some implementations, system 220 may alternatively utilize probe 22 described above.

Communication interface 228 of probe 222 comprises an interface by which probe 222 communicates with host 225. In one implementation, communication interface 228 facilitates wireless communication. For example, in one implementation, communication interface 228 comprises a wireless antenna. In another implementation, communication interface may comprise optical communication technology, such as an infrared transmitter. In another implementation, communication interface 228 facilitates a wired communication such as through a cable. For example, communication interface 228 may comprise a USB port or other communication port.

Host 225 comprises a device in communication with probe 222 which is configured to direct the operation of probe 222. Host 225 further stores and/or presents imaging results from probe 222. Host 225 comprises communication interface 230, input 231, processor 232, input 234, output 233 and memory 234. Communication interface 230 comprises an interface by which host 225 communicates with probe 222. In one implementation, communication interface 230 facilitates wireless communication. For example, in one implementation, communication interface 230 comprises a wireless antenna. In another implementation, communication interface may comprise optical communication technology, such as an infrared transmitter. In another implementation, to communication interface 230 facilitates a wired communication such as through a cable. For example, communication interface may comprise a USB port or other communication port.

Input 231 comprises one or more input devices by which a person may enter commands, selections or data into system 220. Examples of input 231 comprise, but are not limited to, a keyboard, mouse, a touchpad, touchscreen, microphone with speech recognition programming, keypad, pushbuttons, slider bars and the like. As will be described hereafter, input 231 enables the input of mode preferences for transducers 24 and for the display of information on output 233.

Processor 232 is similar to processor 32. Processor 232 comprise one or more processing units configured to generate control signals in response to sensed signals received by sensors 26 (or transducers 24 serving at sensors 26) according to instructions contained in memory 234. One implementation, memory 234 comprises a non-transient computer-readable medium providing a persistent storage for such instructions or code. In one implementation, memory 234 may comprise software. In another implementation, memory 234 and associated portions of processor 232 may comprise an application-specific integrated circuit (ASIC).

Output 233 comprises one or more devices by which the imaging results from probe 222 may be presented for viewing. In one implementation, output 233 comprises a display screen. In another implementation, 233 comprises a printing device. In one example, output 233 may comprise a touchscreen serving as an input, facilitating omission of input 231.

Memory 234 is similar to memory 34 in that memory 234 includes detection module 38 and activation module 40 (described above). As shown by FIG. 3, memory 234 additionally stores selectable or available operation presets or modes 244D, 244B, 244C, 244D, 244E and 244G (collectively referred to as modes 244) and mode module 246. Operation modes 244 comprise sets or groups of settings for transducers 24 and for output 233 which are stored in memory 234. Each of operation modes 244 is associated with one of transducers 24. For example, operation modes 244A, 244B and 244C are each associated with transducer 24A while operation modes 244D, 244E and 244F are each associated with transducer 24B.

Operation modes 244A, 244B and 244C each comprise a different group of settings for use by system 220 when processor 232 indicates that transducer 24A has been positioned for imaging (based on information from sensors 26 and instructions according to detection module 38). Each operation mode 244A, 244B and 244C may designate different operation settings for transducer 24A. For example, each of operation modes 244A, 244B, 244C may designate use of a different particular frequency (different penetration or depth) or a particular angle setting for transducer 24A. In one implementation, each operation mode 244A, 244B and 244C may additionally designate a different operation setting for the processing of image signals for output 233. For example, each operation mode 244A, 244B and 244C may designate a different time gain compensation (TGC) to differently amplify echoes to address increased attenuation experienced as penetration depths increase. Each operation mode 244A, 244B and 244C may designate a different particular frame rate for visibly presenting imaging results. Some frame rates may better illustrate features of the imaged objects, for example, the imaging of cardiac blood flow.

Operation modes 244D, 244E and 244F each comprise a different group of settings for use by system 220 when processor 232 indicates that transducer 24B has been positioned for imaging (based on information from sensors 26 and instructions according to detection module 38). Each operation mode 244D, 244E and 244F may designate different operation settings for transducer 24B. For example, each of operation modes 244D, 244E and 244F may designate use of a different particular frequency (different penetration or depth) or a particular angle setting for transducer 24B. In one implementation, each operation mode 244D, 244E and 244F may additionally designate a different operation setting for the processing of image signals for output 233. For example, each operation mode 244D, 244E and 244F may designate a different time gain compensation (TGC) to differently amplify echoes to address increased attenuation experienced as penetration depths increase. Each operation mode 244D, 244E and 244F may designate a different particular frame rate for visibly presenting imaging results. Some frame rates may better illustrate features of the imaged object, for example, the imaging of cardiac blood flow. Each of such settings for transducer 24B may be distinct from settings for transducer 24A.

Mode module 246 comprises a non-transient computer readable program or code stored in memory 234 and configured to direct processor 232 to in the selection and implementation of one of operation modes 244 upon the determination of which of transducers 24 is about to be physically coupled, or is physically coupled, to tissue overlying the target or object to be imaged. In one example implementation, mode module 246 directs processor 232 to present the various operation modes 244 for each of transducers 24 upon display of output 233 and further directs processor 232 to prompt a person or caretaker to select which of the operation modes 244 is to be assigned to which of the transducers 24. Mode module 246 is further configured to store such selections in memory 234. Upon the subsequent determination by processor 232 that a particular transducer 24 is about to be physically coupled to tissue for imaging or has been physically coupled to tissue for imaging, mode module 246 directs processor 232 to consult memory 234 to retrieve and implement the particular operation mode 244 associated with the particular transducer.

Mode module 246 and the various available operation modes 244 allow a caretaker to customize system 220 to best serve the most prevalent uses of probe 222 and transducers 24. For example, if the caretaker frequently uses transducer 24A for imaging a particular target or object (at a particular depth) and having particular characteristics, the caretaker may assign and store an operation mode best suited for the most prevalent use of transducer 24A, an operation mode 244 having the most appropriate frequency, TGC, angle and the like. The caretaker may also assign and store an operation mode 244 for the most prevalent use of transducer 24A that best displays the results of the imaging on output 233. For example, an operation mode may be selected that has the most appropriate frame rate, magnification, format, selected information being displayed and the like. The same can be done for transducer 24B. Such operation mode settings will then be automatically initiated based upon the detected positioning of the transducers 24.

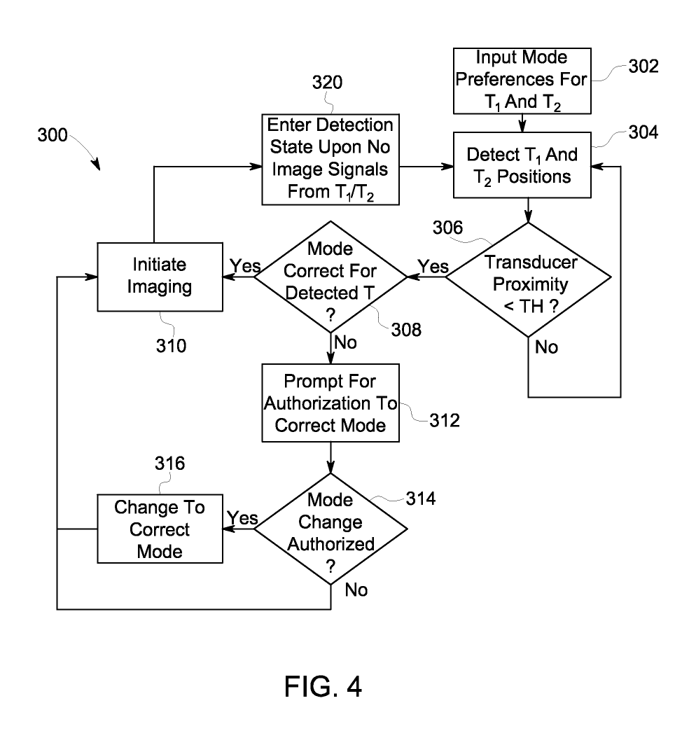

In one implementation, mode module 246 may further be configured to direct processor 232 to provide a graphical user interface on the display of output 233, allowing a caretaker to input and create a new customized operation mode 244 which may be added to the existing operation mode 244 for subsequent assignment and use. In one implementation, mode module 246 is further configured to facilitate comparison of an operation mode currently being used with the transducer being readied for use. As will be described with respect to method 300 shown in FIG. 4, such comparison may reveal that less than optimal or undesirable mode is currently in place, wherein the caretaker may be prompted to authorize changing of the current operation mode to an operation mode assigned to a particular transducer.

FIG. 4 is a flow diagram illustrating an example method 300 that may be carried out by system 220 of FIG. 3. As indicated by step 302, processor 232, following instructions from mode module 246, prompts a caretaker on the display of output 233 to input operation mode preferences for each of transducers 24. Such assignments or selections are stored in memory 234.

As indicated by step 304, detection module 38 directs processor 232 to begin detecting positioning for each of transducers 24 (as discussed above with respect to step 104 in FIG. 2). As indicated by step 306, this sensing or detection of the positioning of transducers 24 continues until processor 232 receives position data from sensors 26 (or from transducers 24 where transducer 24 serve as sensors 26) indicating that one of transducers 24 satisfies its associated positioning threshold (as described above with respect to steps 106 and 108).

As indicated by step 308, upon one of transducers 24 satisfying its associated position threshold TH, mode module 246 directs processor 232 to compare any existing mode currently operating on system 220 to a preferred or assigned operation mode 244 stored in memory 234. As indicated by step 310, if the current mode of operation for system 220 (possibly remaining from the last imaging operation using probe 222) is correct, activation module 40 directs processor 232 to initiate imaging as discussed above with respect to steps 110 or 120 in FIG. 2.

Alternatively, as indicated by step 312, if the current mode of operation for system 220 is incorrect (not matching the mode of operation 244 assigned to the particular transducer) given the particular transducer identified as satisfying the position threshold TH, mode module 246 directs processor 232 to prompt a caretaker for authorization to correct the mode of operation. As indicated by step 314, if such authorization is granted (using input 231), mode module 246 directs processor 232 to change the mode of operation (shown in step 316).

If such authorization is not granted, mode module 246 directs processor 232 to initiate imaging per step 310. Once imaging has been initiated on one of transducers 24, section module 38 directs processor 232 to periodically determine whether image signals are being received from either of transducers 24. In one implementation, such position detection may be dormant while imaging signals are being received from at least one of transducers 24 to preserve power and computing resources. However, as indicated by step 320, upon processor 232 determining that image signals of an underlying target or object are no longer being received for predefined minimum period of time (as discussed above with respect to step 102), based upon a comparison of echo signals to predefined thresholds corresponding to the sensing of underlying target or object), processor 232 may once again enter a position detection state, wherein the positions of transducers are detected per step 304.

In other implementations, steps 312 and 314 may be omitted, wherein the mode of operation is automatically changed without a prompt for authorization. For example, upon detection of transducer 24A being within sufficient proximity to tissue overlying the object to be imaged, system 220 may automatically and without further authorization or input, initiate operation mode 244A (previously assigned to transducer 24A in step 302). As a result, as schematically shown in FIG. 3, processor 232 generates control signals causing transducer 24A to operate using the settings of operation mode 244A (M1)(using designated frequency, angle and the like). Processor 232 further processes such signals received from probe 222 and displays imaging results on the display of output 233 also using the settings of operation mode 244A (M1) (using designated TGC, frame rate, magnification, format, the display of particular information and the like).

Should the caretaker position probe 222 to alternatively position transducer 24B in close proximity or physical coupling with the tissue overlying the object to be imaged, transducer 24A will be removed from tissue, changing characteristics of echo signals such that the signal no longer indicate imaging is taking place. As a result, system 220 once again enters the detection mode per step 302. In response to processor 232 detecting that transducer 24B is satisfying the position threshold TH, mode module 246 automatically changes to the operation mode 244B (assigned in this example to transducer 24B). Activation mode 40 causes processor 232 to initiate imaging per step 310. The switching to mode 244B results in transducer 24B using the settings of operation mode 244B (M1) (using designated frequency, angle and the like). Processor 232 further processes such signals received from probe 222 and displays imaging results on the display of output 233 also using the settings of operation mode 244B (M2) (using designated TGC, frame rate, magnification, the display of particular information and the like).

In one implementation, mode module 246 and detection module 38 may cooperate to implement individual settings of the assigned mode of operation 244 at different times based upon the detected positioning of the particular transducer assigned to the mode of operation. For example, in response to receiving signals from sensor 26A (or transducer 24A where transducer 24A is used to detect positioning) indicating that the sensed positioning of transducer 24A has satisfied a first threshold TH₁, mode module 246 may automatically initiate the use of one of the settings, for example, settings for the display of output 233 such as the format of imaging information. In response to receiving signals from sensor 26A (or transducer 24A where transducer 24A is used to detect positioning) indicating that the sensed positioning of transducer 24A has satisfied a second threshold TH₂ (now closer or in physical coupling with the tissue), mode module 246 may automatically initiate the use of one of other settings, for example, a setting directed at the operation of transducer 24A, such as its frequency. Such different position or distance thresholds may be selected or input by the caretaker when creating an operation mode or preset 244.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An apparatus comprising:
a handheld ultrasound probe;
a first transducer provided by the probe;
a second transducer provided by the probe, wherein the first transducer and the second transducer are spaced so as to be alternately coupled to skin; and
a controller to automatically initiate emission of ultrasound pulses by one of the first transducer or the second transducer based on positioning of said one of the first transducer and the second transducer to tissue overlying an object to be imaged.

2. The apparatus of claim 1, wherein the controller is configured to automatically initiate the emission of ultrasound pulses based upon a detected proximity of one of the first transducer and the second transducer to the skin.

3. The apparatus of claim 2, wherein the controller is configured to automatically initiate emission of ultrasound pulses in response to one of the first transducer and the second transducer being coupled to the skin.

4. The apparatus of claim 2, wherein the controller is configured to automatically initiate emission of ultrasound pulses in response to one of the first transducer and the second transducer being with in a predefined distance from the skin, yet spaced from the skin.

5. The apparatus of claim 1, wherein the first transducer is configured to emit ultrasound pulses along a first line in a first direction and wherein the second transducer is configured to emit ultrasound pulses along a second line in a second direction nonparallel to the first direction.

6. The apparatus of claim 1, wherein the first transducer is configured to emit ultrasound pulses along a first line and a first direction and wherein the second transducer is configured to emit ultrasound pulses along a second line in a second direction opposite to the first direction.

7. The apparatus of claim 1, wherein the controller is configured to automatically switch emissions of ultrasound pulses from the first transducer to the second transducer in response to a determination by the controller that the second transducer has been coupled to the tissue.

8. The apparatus of claim 1, wherein the controller is configured to automatically switch emissions of ultrasound pulses from the first transducer to the second transducer in response to a determination by the controller that the second transducer is about to be coupled to the tissue.

9. The apparatus of claim 1 further comprising at least one sensor to detect coupling of the first transducer or the second transducer to the tissue.

10. The apparatus of claim 9, wherein the at least one sensor is selected from a group of sensors consisting of a thermistor, an optical sensor, a capacitive sensor and a pressure sensor.

11. The apparatus of claim 9, wherein the at least one sensor comprises the controller and the first transducer, wherein the controller is configured to generate control signals causing the first transducer to periodically emit bursts of ultrasound pulses and to sense echoes from the bursts to detect coupling of the first transducer to the tissue.

12. The apparatus of claim 11, wherein the at least one sensor further comprises the second transducer, wherein the controller is configured to generate control signals causing the second transducer to periodically emit bursts of ultrasound pulses and to sense echoes from the bursts from the second transducer to detect coupling of the second transducer to the tissue.

13. The apparatus of claim 1 further comprising a display, wherein the controller is further configured to:
automatically initiate a first one setting in response to a sensed positioning of the first transducer with respect to the tissue; and
automatically initiate a second one of the plurality of available settings in response to a sense positioning of the second transducer with respect to the tissue.

14. The apparatus of claim 13, wherein the controller is configured to:
detect which of the first transducer and the second transducer being used;
compare current settings of the apparatus with the transducer detected as being used; and
input for a change in the settings based upon the comparison.

15. The apparatus of claim 13, wherein the first transducer is configured to transmit a first range of ultrasound frequencies and wherein the second transducer is configured to emit a second range of ultrasound frequencies different than the first range of ultrasound frequencies.

16. A method comprising:
imaging an object by emitting ultrasound pulses and sensing reflections of the ultrasound pulses using a first transducer coupled to tissue while a second transducer is uncoupled from the tissue;
sensing positioning of the second transducer with respect to the tissue; and
automatically initiating anatomical imaging by the second transducer based upon the sensed positioning of the second transducer with respect to the tissue.

17. The method of claim 16 further comprising automatically terminating the anatomical imaging with the first transducer in response to initiation of imaging by the second transducer.

18. The method of claim 16, wherein the first transducer faces in a first direction and wherein the second transducer faces in a second direction opposite to the first direction.

19. An apparatus comprising:
a non-transient computer-readable medium containing code configured to direct a processor to:
generate control signals directing imaging by emitting ultrasound pulses and sensing reflections of the ultrasound pulses using a first transducer coupled to tissue while a second transducer is uncoupled from the tissue;
generate control signals resulting from positioning of the second transducer with respect to the skin; and
automatically initiating imaging by the second transducer based upon the sensed positioning of the second transducer with respect to the tissue.

20. The apparatus of claim 19, wherein the code is configured to direct a processor to automatically terminate the anatomical imaging with the first transducer in response to initiation of object imaging by the second transducer.
